(19) United States
(12) Patent Application Publication
(10) Pub. No.: US 2008/0021239 A1
(43) Pub. Date: Jan. 24, 2008

(54) Process for Producing (Meth)Acrylic Acid or (Meth)Acrolein

(75) Inventors: Yasushi Ogawa, Mie (JP); Shuhei Yada, Mie (JP); Yoshiro Suzuki, Mie (JP); Kenji Takasaki, Mie (JP)

Correspondence Address: OBLON, SPIVAK, MCCLELLAND MAIER & NEUSTADT, P.C., 1940 DUKE STREET, ALEXANDRIA, VA, 22314, US

(73) Assignee: Mitsubishi Chemical Corporation, Minato-ku, Tokyo (JP)

(21) Appl. No.: 11/596,366

(22) PCT Filed: Nov. 5, 2004
(86) PCT No.: PCT/JP04/16788

§ 371 Date: Mar. 23, 2007

(30) Foreign Application Priority Data
May 14, 2004 (JP) 2004-144509

(51) Int. Cl. C07C 45/33

(52) U.S. Cl. 562/512.2; 568/469.9

(57) ABSTRACT

The object of the invention is to provide a method for producing (meth)acrylic acid or (meth)acrolein from at least one of propylene, propane, isobutylene and (meth)acrolein as a raw material by carrying out its gas phase catalytic oxidation reaction with molecular oxygen or a molecular oxygen-containing gas using a multi-tubular reactor, which is a method that can be operated more safely by feeding a gas prepared in advance by mixing the raw material with molecular oxygen or a molecular oxygen-containing gas safely into the reactor by controlling generation and propagation of fire. The invention is a method for producing (meth)acrylic acid or (meth)acrolein, characterized in that a mixed gas formed by mixing the aforementioned raw material with the aforementioned molecular oxygen or a molecular oxygen-containing gas is fed into the aforementioned reactor via a piping equipped with a flame arrester.

### TECHNICAL FIELD

This invention relates to a method for producing (meth)acrylic acid or (meth)acrolein markedly safely from at least one of propylene, propane, isobutylene and (meth)acrolein as a raw material by carrying out gas phase catalytic oxidation of the raw material with molecular oxygen using a multi-tubular reactor.

### BACKGROUND ART

(Meth)acrylic acid and (meth)acrolein are produced by a gas phase catalytic oxidation reaction in which propylene, propane, isobutylene or (meth)acrolein as a raw material is allowed to contact with molecular oxygen or a molecular oxygen-containing gas in the presence of a composite oxide catalyst. In addition, this gas phase catalytic oxidation reaction is generally carried out using a multi-tubular reactor having two or more reaction tubes equipped with a catalyst layer.

As a matter of course, it is required that the product of interest is obtained by safe operation in such a reaction system.

According to the aforementioned reaction system, a method in which the raw material and molecular oxygen or molecular oxygen-containing gas such as air are mixed in advance, and the thus formed mixed gas is fed into a reactor, is general. On the other hand, the composition of this mixed gas is prepared as outside of the range of explosion. Patent Reference 1 (JP-B-58-011247) proposes to complete the mixing process instantly, because a composition sometimes enters into the range of explosion during the process of mixing an organic gas with oxygen. In addition, Patent Reference 2 (JP-A-2002-53519) proposes to carry out normal operation in starting up an oxidation reactor, by changing the operation conditions while avoiding formation of the range of explosion. Though these proposals are respectively superior, a demand for more safety is strong.

### DISCLOSURE OF THE INVENTION

The object of the invention is to provide a method for producing (meth)acrylic acid or (meth)acrolein from at least one of propylene, propane, isobutylene and (meth)acrolein as a raw material by carrying out its gas phase catalytic oxidation reaction with molecular oxygen or a molecular oxygen-containing gas using a multi-tubular reactor, which is a method that can carry out more safe operation by safely introducing a gas prepared by mixing the raw material with molecular oxygen or a molecular oxygen-containing gas in advance into the reactor while controlling break out and spread of fire.

The invention is a method for producing (meth)acrylic acid or (meth)acrolein from at least one of propylene, propane, isobutylene and (meth)acrolein as a raw material by carrying out its gas phase catalytic oxidation reaction with molecular oxygen or a molecular oxygen-containing gas using a multi-tubular reactor, characterized in that a mixed gas of the aforementioned raw material and the aforementioned molecular oxygen or a molecular oxygen-containing gas is fed into the aforementioned reactor via a piping equipped with a flame arrester, and the object of the invention was achieved by this method.

### BRIEF DESCRIPTION OF THE DRAWINGS

FIG. 1 is a schematic sectional view showing an embodiment of the multi-tubular heat exchange type reactor to be used in the gas phase catalytic oxidation method of the invention,

FIG. 2 is a schematic illustration showing an embodiment of baffles of the multi-tubular heat exchange type reactor to be used in the invention,

FIG. 3 is a schematic illustration showing another embodiment of baffles of the multi-tubular heat exchange type reactor to be used in the invention,

FIG. 4 is a schematic sectional view showing another embodiment of the multi-tubular heat exchange type reactor to be used in the gas phase catalytic oxidation method of the invention,

FIG. 5 is an enlarged schematic sectional view of an intermediate tube plate which divides the shell of the multi-tubular heat exchange type reactor shown in FIG. 4, and

FIG. 6 is a schematic illustration showing an example of flame arrester.

In this connection, the reference numerals 1b and 1c in the drawings are reaction tubes, 2 is a reactor, 3a and 3b are circular conduits, 3a′ and 3b′ are circular conduits, 4a is a product discharging outlet, 4b is a raw material feeding inlet, 5a and 5b are tube plates, 6a and 6b are perforated baffles, 6a′ and 6b′ are perforated baffles, 7 is a circulation pump, 8a and 8a′ are heat medium supplying lines, 8b and 8b′ are heat medium discharging line, 9 is an intermediate tube plate, 10 is a heat shielding plate, 11, 14 and 15 are thermometers, 12 is a stagnation space, 13 is a spacer rod, 21 and 21′ are flanges for connecting to piping, 22 is a quenching element, and 23 is a reinforcing plate.

### BEST MODE FOR CARRYING OUT THE INVENTION

The following describes the invention in detail.

As described in the foregoing, the invention is a method for producing (meth)acrylic acid or (meth)acrolein from at least one compound to be oxidized among propylene, propane, isobutylene and (meth)acrolein as a raw material by carrying out its gas phase catalytic oxidation reaction with molecular oxygen or a molecular oxygen-containing gas using a fixed bed multi-tubular reactor, characterized in that the aforementioned raw material and the aforementioned molecular oxygen or a molecular oxygen-containing gas are mixed in advance, and the thus formed mixed gas is fed into the aforementioned reactor via a piping equipped with a flame arrester. By the arrangement of a flame arrester, even when a flame is generated for some reason in the aforementioned piping which supplies the raw material into the reactor, the flame is quenched by the flame arrester.

According to the invention, the flame arrester has the same meaning as a flame propagation preventing device. Details on the flame propagation preventing device are described in Anzen Kogaku Koza 2 Bakuhatsu (Safety Engineering Series 2 Explosion), pp. 101-315, published on Mar. 1, 1983, published by Kaibundo Shuppan.

A wire netting type is desirable as the flame arrester to be used in the invention.

In an example of the wire netting type flame arrester, from 1 to 30 (more preferably from 3 to 10) wire nettings having a mesh size of from 10 to 150 (more preferably from 30 to 80) are superimposed. There is a tendency to decrease quenching effect when the mesh size is less than 10. Also, when it exceeds 150, pressure loss becomes large thus causing a tendency to reduce the yield of acrylic acid. A combination of flame arresters of from 30 to 80 mesh is desirable for increasing quenching effect and reducing pressure loss. Regarding the superimposing number of wire nettings, the aforementioned number is desirable because of the large effect in increasing quenching effect and reducing pressure loss.

The wire nettings to be superimposed may have a space of preferably from 0 to 20 mm, more preferably from 0 to 10 mm. Wire nettings having different mesh sizes may also be superimposed.

When wire nettings are superimposed by closely contacting them, it is desirable to superimpose in such a manner that strands of the adjoining wire nettings mutually have an angle of within the range of preferably from 30 to 150 degree, more preferably from 45 to 90 degree, for increasing the quenching effect.

In practically designing a wire netting type flame arrester, the following formula (1) described in Health and Safety Executive: Flame Arrester and Explosion Reliefs, Health and safety series booklet HS (G) 11, Her Majesty's Stationary Office, London (1980) will be helpful.
v=0.5 aL/D² (1)
(In the formula, v is a critical flame speed (ft/s), a is a ratio of the total area of openings of a wire netting to the total exposed area of the wire netting, L is thickness of the wire netting (the double of strand diameter) (in), and D is opening of the wire netting (in).)

The number of wire nettings to be superimposed is not particularly limited, but the quenching ability is increased with the number of even 5 or more when they are fine mesh wire nettings (cf. the aforementioned “Safety Engineering Series 2 Explosion”).

In addition, since a wire netting type flame arrester has a possibility of generating “clogging”, it is desirable to carry out its checking or maintenance work such as its periodical exchange.

Schematic illustration of an example of the flame arrester is shown in FIG. 6. The flame arrester of FIG. 6 is attached to the mixed gas piping with the left and right side flanges 21 and 21′ such that it can be inserted into the channel of mixed gas, and the quenching element 22 constructed by a wire netting is arranged at a right angle toward the direction of flame shown by an arrow and fixed by being protected with a reinforcing plate 23, so that it functions to check propagation of flame even in case of explosion. In this connection, FIG. 6 is a typical example of the flame arrester, though not limited thereto.

The flame arrester is arranged on the piping which supplies the mixed gas from a mixer for mixed gas formation into the reactor. Regarding the suitable arranging site between the mixer and the reactor, it is desirable to arrange it at a position close to the mixer outlet when the distance from the mixer to the reactor is relatively short and the passing time of gas is within 2 seconds, and when the distance is relatively long and the passing time of gas becomes 2 seconds or more, or when it is 2 seconds or less but the reaction gas is constituted by a composition of within the range of explosion, it is desirable to fill a space from the mixer to the reaction tube inlet inside the reactor (tube plate of the reactor) with wire nettings as the flame arrester. Two or more flame arresters may be arranged on the piping, and from 1 to 10 of them are generally arranged.

The following describes the reaction system, reactor, catalyst and others to be used for producing (meth)acrylic acid or (meth)acrolein by carrying out gas phase catalytic oxidation reaction of the mixed gas supplied, as described in the above, into the reactor through the piping equipped with a flame arrester.

(Reaction System)

Typical examples of the reaction system in the industrialized acrolein and acrylic acid production method include a one pass system, an unreacted gas recycling system and an exhaust gas recycling system described in the following, and the reaction systems including these three systems are not restricted in the invention.

(1) One Pass System:

This system is a method in which propylene, air and steam are mixed and supplied and converted mainly into acrolein and acrylic acid in the former stage reaction, and this outlet gas is supplied to the latter stage reaction (mainly, acrolein is converted into acrylic acid) without separating from the product. In this case, a method in which air and steam necessary for carrying out the reaction in the latter stage reaction are supplied to the last stage reaction, in addition to the former stage outlet gas, is also general.

(2) Unreacted Gas Recycling System:

This system is a method in which a reaction product gas containing acrylic acid obtained by the latter stage reaction is fed into an acrylic acid collecting device where acrylic acid is collected as an aqueous solution, and a part of the unreacted gas is recycled by supplying a part of the acrylic acid collecting device side exhaust gas containing unreacted propylene or propane into the former stage reaction.

(3) Exhaust Gas Recycling System:

This system is a method in which a reaction product gas containing acrylic acid obtained by the latter stage reaction is fed into an acrylic acid collecting device where acrylic acid is collected as an aqueous solution, whole volume of the exhaust gas of the acrylic acid collecting device side is oxidized by combustion to convert the unreacted gas and the like contained therein mainly into carbon dioxide and water, and a part of the thus obtained combustion exhaust gas is added to the former stage reaction.

In general, a multi-tubular reactor is used when it is necessary to increase productivity of the reactor by protecting a catalyst and keeping performance of the catalyst at a high level trough strict management of the reaction temperature of the catalyst, when the heat of reaction is extremely large like the case of an oxidation reaction.

In recent years, production of acrylic acid from propane or propylene and methacrylic acid from isobutylene (generally expressed as (meth)acrylic acid) has been sharply expanding accompanied by the increase of its demands, and a large number of plants have been constructed in the world and the production scale of plants has also been expanded to one hundred thousand tons or more per plant per year. Because of the expansion of the plant production scale, it is under the necessity of expanding the production amount per one oxidation reactor and, as a result, load of the gas phase catalytic oxidation reactor for propane, propylene or isobutylene is increased. Accompanied by this, a more high performance and extremely safe oxidation reactor is in demand.

The invention is to a gas phase catalytic oxidation method for obtaining (meth)acrolein or (meth)acrylic acid by using propylene, propane, isobutylene or (meth)acrolein, or a mixture thereof, as the substance to be oxidized (raw material) and carrying out its gas phase catalytic oxidation with a molecular oxygen-containing gas. (Meth)acrolein, (meth)acrylic acid or both of them are obtained from propylene, propane and isobutylene. Also, (meth)acrylic acid is obtained from (meth)acrolein.

According to the invention, the “process gas” means gases concerned in the gas phase catalytic oxidation reaction, such as a substance to be oxidized as the raw material gas, a molecular oxygen-containing gas, an obtained product and the like. In addition, the “raw material” has the same meaning as the substance to be oxidized.

(Raw Material Gas Composition)

According to the oxidation reactor to be used in the gas phase catalytic oxidation, a mixed gas of at least one substance to be oxidized among propylene, propane, isobutylene and (meth)acrolein as the raw material gas, a molecular oxygen-containing gas and steam is mainly fed into the oxidation reactor from a piping equipped with a fame arrester. The composition of them is outside the range of explosion.

According to the invention, concentration of the substance to be oxidized in the mixed gas is from 4 to 10% by mole, and oxygen is from 1.5 to 2.5 moles and steam is from 0.8 to 5 moles per mole of the substance to be oxidized. The introduced mixed gas undergoes the reaction in the presence of an oxidation catalyst filled in the oxidation reactor.

(Multi-Tubular Reactor)

The gas phase catalytic oxidation reaction of the invention which uses a multi-tubular reactor is a method broadly used in producing (meth)acrylic acid or (meth)acrolein from at least one substance to be oxidized among propylene, propane, isobutylene and (meth)acrolein using molecular oxygen or a molecular oxygen-containing gas in the presence of a composite oxide catalyst.

The multi-tubular reactor to be used in the invention is a generally industrially used one and has no particular limitation.

The following describes an embodiment of the multi-tubular reactor to be used in the invention based on FIG. 1 to FIG. 5.

(FIG. 1)

FIG. 1 is a schematic sectional view for showing an embodiment of the multi-tubular heat exchange type reactor to be used in the gas phase catalytic oxidation method of the invention.

Reaction tubes 1b and 1c are fixed to tube plates 5a and 5b and arranged in a shell 2 of the multi-tubular reactor. The raw material supply port as an inlet of raw material gas of the reaction and the product discharging outlet as an outlet of the product are 4a or 4b. Flow direction of the process gas may be in any direction when the process gas and heat medium are counter current, but since flow direction of the heat medium inside of the reactor shell is shown in FIG. 1 by an arrow as ascending current, 4b is the raw material supply port. A circular conduit 3a for introducing the heat medium is arranged on the periphery of the reactor shell. The heat medium pressured by a circulation pump 7 of the heat medium ascends inside of the reactor shell from the circular conduit 3a and then is returned to the circulation pump from a circular conduit 3b through its turning of flow direction due to the alternate arrangement of two or more of a perforated baffle 6a having an opening at around the central part of the reactor shell and a perforated baffle 6b arranged in such a manner that it has an opening between a peripheral part of the reactor shell. A part of the heat medium which absorbed the heat of reaction is cooled by a heat exchanger (not shown in the drawing) through a discharging tube arranged on the upper part of the circulation pump 7 and again introduced into the reactor from a heat medium supplying line 8a. Adjustment of the heat medium temperature is carried out by adjusting temperature or flow rate of the reflux heat medium supplied from the heat medium supplying line 8a and controlling the thermometer 14.

Though it depends on the performance of the catalyst, temperature control of the heat medium is carried out in such a manner that a difference in the heat medium temperature between the heat medium supplying line 8a and a heat medium discharging line 8b becomes from 1 to 10° C., preferably from 2 to 6° C.

In order to minimize circumferential distribution of the heat medium flow rate, it is desirable to arrange current plates (not shown in the drawing) on the inner shell plate parts of the circular conduits 3a and 3b. A porous plate, a plate having slits or the like is used as the current plate, and rectification is carried out by changing opening area or slit interval of the porous plate in such a manner that the heat medium inflows from the entire circumference at the same flow rate. Temperature in the circular conduit (3a, preferably 3b, too) can be monitored by arranging two or more of a thermometer 15.

The number of baffles to be arranged in the reactor shell is not particularly limited, but it is desirable as usual to arrange 3 baffles (two 6a type and one 6b type). By the presence of these baffles, ascending flow of the heat medium is disturbed so that it turned into the transverse direction against the tube axis direction of the reaction tube, and the heat medium is concentrated into the central part the reactor shell from its peripheral part, turns toward the peripheral part by turning the direction at the opening part of the baffle 6a and then reaches outer casing of the shell. The heat medium is concentrated into the central part by again turning the direction at the periphery of the baffle 6b, ascends the opening part of the baffle 6a, turns toward the periphery along the upper tube plate 5a of the reactor shell, and is recycled in the pump through the circular conduit 3b.

A thermometer 11 is inserted into at least one of the reaction tubes arranged in the reactor and transfers the signal to outside of the reactor, and temperature distribution in the reactor tube axis direction of the catalyst layer is recorded. One or two or more thermometers are inserted into the reaction tube, and from 5 to 20 points of temperatures are measured in the tube axis direction by one thermometer thereof.

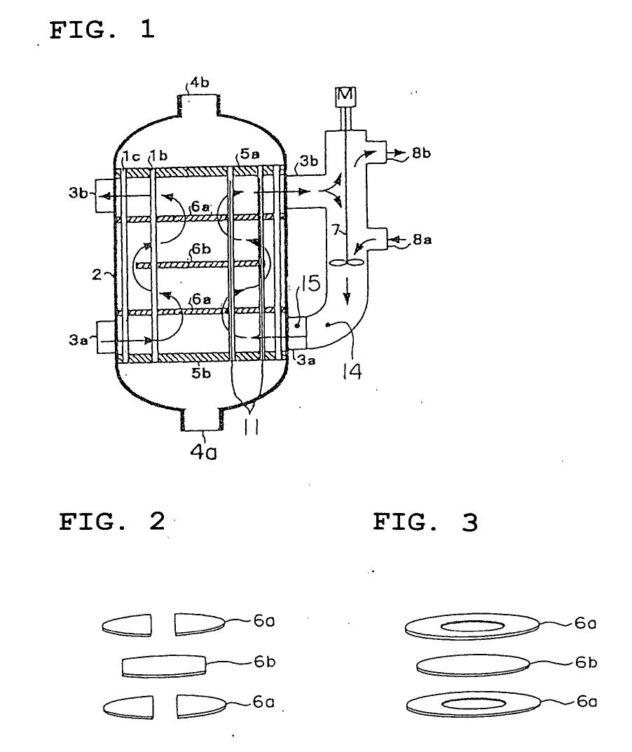

(FIG. 2 and FIG. 3: Baffles)

According to the baffles to be used in the invention, either of the segment type broken disc baffles shown in FIG. 2 or the disc shape baffles shown in FIG. 3 can be employed, with the proviso that they have such a construction that the baffle 6a has an opening part around the central part of the reactor shell, the baffle 6b has an opening between the peripheral part and outer casing of the shell, and the heat medium turns its direction at respective opening parts to prevent bypass flow of the heat medium and change its flow rate. The relationship between flow direction of the heat medium and tube axis of the reaction tube is unchanged in both types of the baffles.

As the general baffles, particularly the disc shape baffles of FIG. 3 are frequently used. The central part opening area of the baffle 6a is preferably from 5 to 50%, more preferably from 10 to 30%, of the sectional area of the reactor shell. The opening area between the baffle 6b and the shell plate of reactor shell is preferably from 5 to 50%, more preferably from 10 to 30%, of the sectional area of the reactor shell. When opening ratio of the baffles (6a and 6b) is to small, channel of the heat medium becomes long, so that the pressure loss between circular conduits (3a and 3b) is increased and the motive power of the heat medium circulation pump 7 becomes large. When opening ratio of the baffles is too large, the number of reaction tube (1c) is increased.

The arranging intervals of respective baffles (interval between baffles 6a and 6b and interval between baffle 6a and tube plates 5a and 5b) are regular intervals in most cases, but not necessarily being equal distances. It is desirable to arrange them in such a manner that necessary flow rate of the heat medium can be ensured, which is determined by the heat of oxidation reaction generated inside of the reaction tubes, and pressure loss of the heat medium can be lowered.

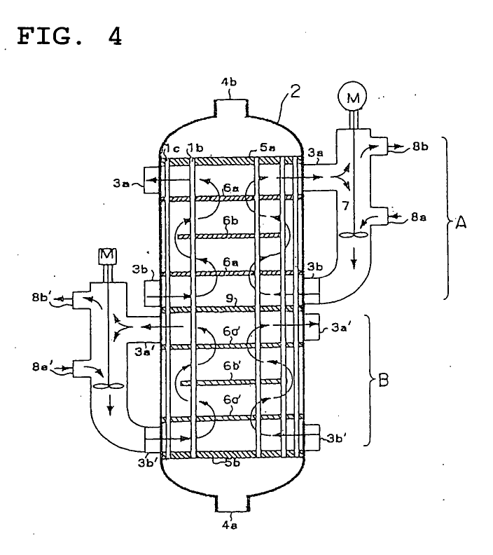

(FIG. 4)

FIG. 4 shows a schematic sectional view of a multi-tubular reactor wherein shell of the reactor is divided with an intermediate tube plate 9, and a method which uses this is also included in the gas phase catalytic oxidation method of the invention. Different heat media are circulated and controlled at different temperatures in the divided spaces. The raw material gas may be supplied from either 4a or 4b, but since flow direction of the heat medium inside of the reactor shell is shown in FIG. 4 by an arrow as ascending current, 4b is the raw material supply port where flow of the raw material process gas becomes counter current with the flow of heat medium. The raw material gas supplied from the raw material feeding inlet 4b successively undergoes the reaction in the reaction tubes of the reactor.

Since heat media having different temperature are present in the upper and lower areas (area A and area B in FIG. 4) of the reactor divided with the intermediate tube plate 9 according to the multi-tubular reactor shown in FIG. 4, inside of each reaction tube is divided into 1) a case in which the same catalyst is evenly filled and the reaction is carried out by changing temperature at the raw material gas inlet and outlet of the reaction tube, 2) a case in which a catalyst is filled in the raw material gas inlet, but in order to rapidly cool the reaction product, the catalyst is not filled in the outlet part to form an empty tube or an inert substance having no reaction activity is filled therein, and 3) a case in which different catalysts are filled in the raw material gas inlet part and outlet part, but in order to rapidly cool the reaction product, the catalyst is not filled between them to form an empty tube or an inert substance having no reaction activity is filled therein.

For example, propylene, propane or isobutylene is supplied as a mixed gas with a molecular oxygen-containing gas from the raw material feeding inlet 4b into the multi-tubular reactor shown in FIG. 4 to be used in the invention and firstly converted into (meth)acrolein at the first step for the former stage reaction (area A of the reaction tube), and then (meth) acrylic acid is produced at the second step for the latter stage reaction (area B of the reaction tube) by oxidizing (meth)acrolein. Different catalysts are filled in the first step part of the reaction tube (to be referred also to as “former stage part” hereinafter) and the second step part (to be referred also to as “latter stage part” hereinafter), and the reactions are carried out under optimum conditions by controlling at respectively different temperatures. It is desirable that an inert substance which is not concerned in the reaction is filled in a part where the intermediate tube plate is present between the former stage part of the reaction tube and the latter stage part.

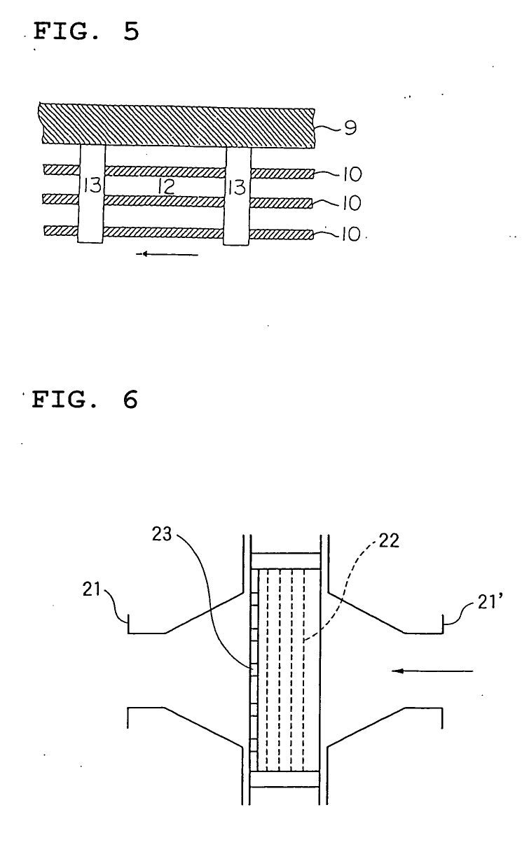

(FIG. 5)

The intermediate tube plate is shown in FIG. 5 by expanding it. The former stage part and the latter stage part are controlled at different temperatures, but when the difference in temperature exceeds 100° C., it becomes impossible to disregard thermal transfer from the high temperature heat medium to the low temperature heat medium, thus showing a tendency to worsen accuracy of the reaction temperature at the low temperature side. In that case, thermal insulation is required for preventing the thermal transfer at the upper or lower side of the intermediate tube plate. FIG. 5 is a case in which an adiabatic plate is used, and the heat medium is filled by arranging 2 or 3 heat shielding plates 10 at a position of about 10 cm downside or upside of the intermediate tube plate, but it is desirable to create an adiabatic effect by forming a stagnation space 12 which has no flow. The heat shielding plate 10 is fixed to the intermediate tube plate 9, for example with a spacer rod 13.

Though flow direction of the heat medium inside of the reactor shell is described as an ascending flow by an arrow in FIG. 1 and FIG. 4, the reverse direction can also be possible in the invention. In deciding direction of circulating flow of the heat medium, it is necessary to avoid a phenomenon in which a gas which may be present in the upper terminals of the reactor shell 2 and circulation pump 7, illustratively nitrogen or the like inert gas, is dragged in the flow of heat medium. In case that the heat medium is ascending flow (FIG. 1), a cavitation phenomenon is found inside the circulation pump 7 when a gas is dragged in the upper part of the circulation pump, causing damage of the pump in the worst case. When the heat medium is descending flow, a dragging phenomenon of a gas occurs in the upper part of the reactor shell, so that a stagnant part of a gas phase is formed in the upper part of the shell, and the upper part of the reaction tube where the gas stagnation part is formed cannot be cooled by the heat medium.

In order to prevent the gas stagnation, it is essential to replace the gas in the gas layer with the heat medium by arranging a degassing line. For this purpose, when the heat medium is ascending flow (FIG. 1), pressure increase in the shell is devised by increasing heat medium pressure of the heat medium supplying line 8a and arranging the heat medium discharging line 8b at a position as upper as possible. It is desirable to arrange the heat medium discharging line on at least upper side of the tube plate 5a.

(Reaction Tube Diameter)

The reaction tube inner diameter which greatly exerts influence upon the gas linear velocity is very important, because inside of the reaction tube containing an oxidation catalyst is a gas phase in the oxidation reactor, and the gas linear velocity is restricted by the resistance of the catalyst so that heat transfer coefficient inside the tube is most small and becomes heat transfer rate-limiting factor.

The reaction tube inner diameter of the multi-tubular reactor according to the invention is influenced by the quantity of heat of reaction and catalyst particle size in the reaction tube, but is preferably from 10 to 50 mm, more preferably from 20 to 30 mm. When the reaction tube inner diameter is too small, amount of the catalyst to be filled is reduced and the number of reaction tubes based on the necessary amount of catalyst is increased, so that a considerable production cost is required due to increased labor at the time of the reactor production, thus worsening industrial economy. On the other hand, when the reaction tube inner diameter is too large, the reaction tube surface area becomes small based on the necessary amount of catalyst, so that the heat transfer area for removing heat of reaction is reduced.

Concomitant items of the invention are described in the following.

(Process for Producing Acrylic Acid or Acrylic Acid Esters)

The following (i) to (iii) and the like can be exemplified as the process for producing acrylic acid.

(i) A process comprising an oxidation step in which gas phase catalytic oxidation of propane, propylene and/or acrolein is carried out, a collecting step in which the acrylic acid-containing gas from the oxidation step is allowed to contact with water, and acrylic acid is collected as an acrylic acid aqueous solution, and an extraction step in which acrylic acid is extracted from this acrylic acid aqueous solution using an appropriate extraction solvent, subsequently separating acrylic acid and the solvent and purifying it by arranging a purification step, further supplying an acrylic acid Michael addition product and a high boiling liquid containing the polymerization inhibitor used in respective steps, as raw materials to a decomposition reaction column to recover valuable substances, and supplying the valuable substances to any one of the steps in and after the collecting step.

(ii) A process comprising an oxidation step in which acrylic acid is produced by carrying out gas phase catalytic oxidation of propylene, propane and/or acrolein, a collecting step in which the acrylic acid-containing gas is allowed to contact with water, and acrylic acid is collected as an acrylic acid aqueous solution, an azeotropic separation step in which this acrylic acid aqueous solution is distilled in an azeotropic separation column in the presence of an azeotropic solvent and crude acrylic acid is recovered from the column bottom, and an acetic acid separation step for removing acetic acid, subsequently carrying out purification by arranging a purification step for removing high boiling impurities, further supplying an acrylic acid Michael addition product after the purification and a high boiling liquid containing the polymerization inhibitor used in respective steps, as raw materials to a decomposition reaction column to recover valuable substances, and supplying the valuable substances to any one of the steps in and after the collecting step.

(iii) A process comprising an oxidation step in which acrylic acid is produced by carrying out gas phase catalytic oxidation of propylene, propane and/or acrolein, a collecting/separation step in which the acrylic acid-containing gas is allowed to contact with an organic solvent to collect acrylic acid as an acrylic acid organic solvent solution, and water, acetic acid and the like are simultaneously removed, a separation step in which acrylic acid is separated from this acrylic acid organic solvent solution, a subsequent step in which a high boiling liquid containing the polymerization inhibitor and organic solvent used in respective production-steps and an acrylic acid Michael addition product, as raw materials to a decomposition reaction column to recover valuable substances, and the valuable substances are supplied to any one of the steps in and after the collecting step, and a step in which the organic solvent is partially purified.

In addition, a polymerization inhibitor is used in the production of acrylic acid which is an easily polymerizable compound, in order to inhibit generation of polymers during the production.

Illustrative examples of the polymerization inhibitor include a copper acrylate, a copper dithiocarbamate, a phenol compound, a phenothiazine compound and the like. Examples of the copper dithiocarbamate include copper dimethyldithiocarbamate, copper diethyldithiocarbamate, copper dipropyldithiocarbamate, copper dibutyldithiocarbamate and the like copper dialkyldithiocarbamates, copper ethylenedithiocarbamate, copper tetramethylenedithiocarbamate, copper pentamethylenedithiocarbamate, copper hexamethylenedithiocarbamate and the like copper cyclic alkylenedithiocarbamates, and copper oxydiethylenedithiocarbamate and the like copper cyclic oxydialkylenedithiocarbamate and the like. Examples of the phenol compound include hydroquinone, methoquinone, pyrogallol, catechol, resorcin, phenol, cresol and the like. Examples of the phenothiazine compound include phenothiazine, bis(α-methylbenzyl)-phenothiazine, 3,7-dioctylphenothiazine, bis(α-dimethyl-benzyl)phenothiazine and the like.

Substances other than the above may be included depending on the process, but it is evident that their kinds do not exert influence upon the invention.

The acrylic acid obtained in this manner is used in various applications. Illustratively, high absorption resins, coagulants, thickeners, acrylate raw materials and the like applications can be exemplified.

While the invention has been described in detail and with reference to specific embodiments thereof, it will be apparent to one skilled in the art that various changes and modifications can be made therein without departing from the spirit and scope thereof.

This application is based on a Japanese patent application filed on May 14, 2004 (Patent Application No. 2004-144509), the entire contents thereof being hereby incorporated by reference.

### INDUSTRIAL APPLICABILITY

According to the method of the invention, a mixed gas of a raw material with molecular oxygen or a molecular oxygen-containing gas is fed into a reactor via a piping equipped with a flame arrester, so that even when composition of the mixed gas enters within the range of explosion at the worst due to operational failure, a flame generated in the piping to the reactor is quenched by the flame arrester, and the flame therefore can be stopped before reaching the reactor. Accordingly, production of (meth)acrylic acid or (meth)acrolein can be carried out markedly safely.

In addition, the production can be carried out safely even when allowed to come near within the range of explosion. Accordingly, production efficiency is improved which is economically advantageous.

1. A method for producing (meth)acrylic acid or (meth)acrolein from at least one of propylene, propane, isobutylene and (meth)acrolein as a raw material by carrying out its gas phase catalytic oxidation reaction with molecular oxygen or a molecular oxygen-containing gas using a multi-tubular reactor,
characterized in that a mixed gas of said raw material and said molecular oxygen or a molecular oxygen-containing gas is fed into said reactor via a piping equipped with a flame arrester.
